SEQUENCE LISTING (5 sequences)

SEQ ID NO: 1 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 2 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 3 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 4 - protein, 295 aa, Homo sapiens [sequence omitted]

SEQ ID NO: 5 - protein, 7 aa, Artificial
  synthetic peptide
QFLGSYL

(19) United States
(12) Patent Application Publication
(10) Pub. No.: US 2011/0200590 A1
(43) Pub. Date: Aug. 18, 2011

(54) Antibodies Directed Against the Myelin Basic Protein Recognising an Epitope of CD64 and Their Use as Immunosuppressants

(76) Inventor: Pasquale Annunziata, Siena (IT)

(21) Appl. No.: 11/916,769

(22) PCT Filed: Jun. 8, 2006
(86) PCT No.: PCT/IT2006/000429

§ 371 Date: Dec. 6, 2007

(30) Foreign Application Priority Data
Jun. 8, 2005 (IT) RM2005A000297

(51) Int. Cl. A61K 39/395; C07K 16/18; A61P 25/00; C07H 21/04; C12N 15/63

(52) U.S. Cl. 424/133.1; 530/389.1; 530/387.3; 530/387.9; 424/172.1; 536/23.53; 435/320.1

(57) ABSTRACT

It is described an antibody, recombinant or synthetic fragments thereof able to recognise and bind at least one epitope of the myelin basic protein. The antibody is also able to recognise also an epitope of the protein of the class of Fc receptors (FcR), namely CD64. Therapeutic applications are also disclosed.

### TECHNICAL FIELD OF THE INVENTION

The present invention relates to human or humanised monoclonal antibodies directed against the myelin basic protein which recognise an epitope of the protein FcRI (CD64), expressed on the outer membrane of monocytes and macrophages, with high affinity for IgG. Moreover, the invention relates to their use as immunosuppressants, in particular in the pathology of multiple sclerosis.

### PRIOR ART

Multiple sclerosis (MS) is an inflammatory disease of the central nervous system, believed to be caused by an attack of the lymphocytes T and B against antigens of myelin which causes demyelination and axonal loss. The clinical course is heterogeneous and it is characterised by the presence of remitting-relapsing or progressive clinical forms (1). 5 to 40% of patients have a benign course at the onset (2, 3) and in any case they can have a long time interval between the first and the second clinical attack, or the subsequent ones, supporting the concept of stable MS. Over 50% of these patients with benign, or otherwise stable forms, exhibit an acceleration of the progression of the illness after 10 years from the onset (4). This data suggest that immunological factors constituting the basis for the benign phase may be lost over time. The identification of such factors could thus provide the bases for new therapeutic treatments based on the use of potentially protective molecules.

The author of the present invention has recently demonstrated that high levels of IgM, but not of IgG, directed against the myelin basic protein (MBP) are significantly associated with a precociously favourable course of MS within the first two years of illness, and then progressively disappear, supporting the hypothesis of an exhaustion of the synthesis of said protective antibodies (5). The author has: a) identified and isolated lymphocyte clones producing anti-MBP monoclonal antibodies and other myelinic proteins from patients with MS and from controls, in relation to the clinical illness course; b) identified that some of said antibodies also recognise an epitope of the protein FcRI, CD64; c) characterise their biological activity in vitro and in a chronic model of experimental autoimmune encephalomyelitis (CEAE) considered an animal model for human MS.

Methods for treating autoimmune diseases using antibodies have been described. For example, WO00/69461 describes a monoclonal antibody, F28C4, which is idiotypic for a T-cell receptor involved in the auto-immunity condition. This antibody is directed against the 1-9-acetyl amino acids of MBP. The patent EP 0.610.446 describes a peptide of a length from 8 to 25 amino acids which is homologous to the amino acid sequence of human MBP from amino acid 61 to amino acid 106, and/or is able to neutralise the anti-MBP.

Traditional immunosuppressants that are used to treat autoimmune diseases and/or immunomediated pathologies of the nervous system all act by an action on the cellular cycle, with potential medium-term oncogenic risk (6). The risk is increased in autoimmune pathologies requiring a long term chronic therapy. Therefore, there is an evident need to identify and develop new immunosuppressive therapies that act with different action mechanism from that of drugs used commonly in therapy and that are potentially less toxic.

### DISCLOSURE OF THE INVENTION

Therefore, an object of the present invention is an antibody or its recombinant or synthetic fragments, able to recognise and bind at least one epitope of the myelin basic protein having the following sequence:

(SEQ ID No 1)
MASQKRPSQR HGSKYLATAS TMDHARHGFL PRHRDTGILD
SIGRFFGGDR GAPKRGSGKD SHHPARTAHY GSLPQKSHGR
TQDENPVVHF FKNIVTPRTP PPSQGKGRGL SLSRFSWGAE
GQRPGFGYGG RASDYKSAHK GFKGVDAQGT LSKIFKLGGR
DSRSGSPMAR R

Preferably, the antibody or its recombinant or synthetic fragments are able to recognise also an epitope of the protein of the class of the Fc receptors (FcR). More preferably, the protein of the class of the receptors Fc (FcR) is the protein FcRI (CD64) having the following sequence:

(Seq ID No 2)
MWFLTTLLLW VPVDGQVDTT KAVITLQPPW VSVFQEETVT
LHCEVLHLPG SSSTQWFLNG TATQTSTPSY RITSASVNDS
GEYRCQRGLS GRSDPIQLEI HRGWLLLQVS SRVFTEGEPL
ALRCHAWKDK LVYNVLYYRN GKAFKFFHWN SNLTILKTNI
SHNGTYHCSG MGKHRYTSAG ISVTVKELFP APVLNASVTS
PLLEGNLVTL SCETKLLLQR PGLQLYFSFY MGSKTLRGRN
TSSEYQILTA RREDSGLYWC EAATEDGNVL KRSPELELQV
LGLQLPTPVW FHVLFYLAVG IMFLVNTVLW VTIRKELKRK
KKWDLEISLD SGHEKKVISS LQEDRHLEEE LKCQEQKEEQ
LQEGVHRKEP QGAT

Yet more preferably, the epitope of the myelin basic protein is comprised in the amino acid region from aa 105 to aa 120 of seq ID 1, preferably it consists of the amino acid region from aa 109 to aa 116 of SEQ ID No 1 and the epitope of the protein CD64 essentially consists of the amino acid region from aa 222 to aa 228 of seq ID 2. In a preferred embodiment, the antibody or its recombinant or synthetic fragments is of human origin or humanised.

An object of the present invention is said antibody or its recombinant or synthetic fragments for medical use.

Another object of the present invention is a pharmaceutical composition for the prevention and/or treatment of autoimmune diseases comprising in therapeutically effective quantities said antibody or its said recombinant or synthetic fragments and appropriate dilutants, and/or excipients and/or vehicles. Preferably, the pharmaceutical composition is used for the prevention and treatment of multiple sclerosis.

An object of the present invention is a nucleic acid encoding for said antibody or its said recombinant or synthetic fragments, a recombinant expression vector comprising said nucleic acid able effectively to express said antibody or its said recombinant or synthetic fragments.

The present invention shall now be described in non limiting examples thereof, with particular reference to the following figures:

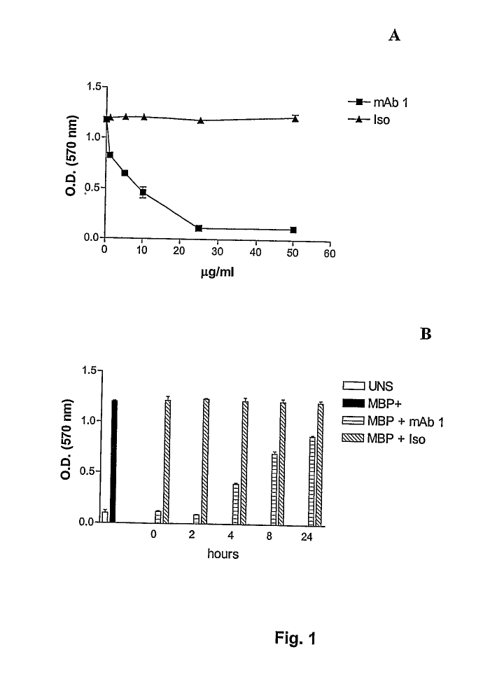

FIG. 1: (A) Concentration curve of the inhibitory activity of the proliferation of human T cell lines sensitised to myelinic proteins of the anti-MBP monoclonal antibody 105-120 (mAb1) and of a human myeloma IgM antibody used as control (Iso). (B) Inhibitory activity of the proliferation of human T cell lines sensitised to myelinic proteins of the anti-MBP monoclonal antibody 105-120 (mAb1). The results are expressed in optical density (O.D.) as mean±standard error (ES) of triplicate cultures of a representative experiment (n=3). UNS=non stimulated human T cell lines; MBP+=human T cell lines sensitised to stimulated MBP; Mab1=anti-MBP monoclonal antibody 105-120; Iso=human myeloma IgM.

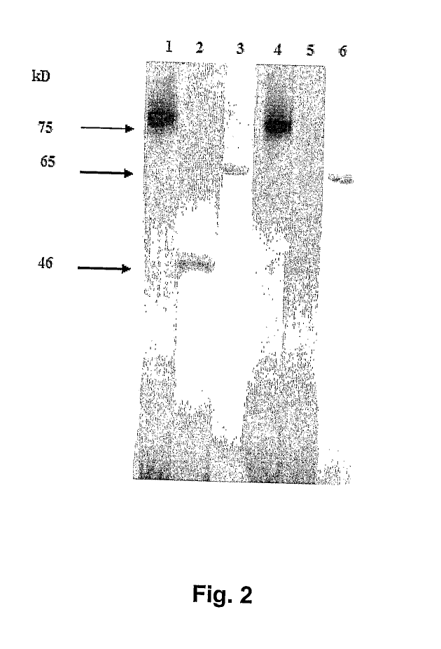

FIG. 2: Protein pattern immunoprecipitated by the anti-MBP monoclonal antibody 105-120. Lines 1-3: human monocytes; lines 4-6: mouse splenocytes; 1 and 4: band 75 Kd immunoprecipitated by the anti-MBP monoclonal antibody 105-120; 2 and 5: band immunoprecipitated by the human myeloma IgM; 3 and 6: band immunoprecipitated by a human Class II anti-MHC monoclonal antibody.

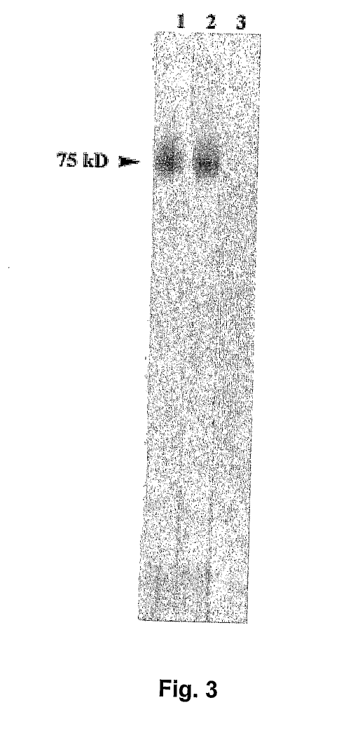

FIG. 3: Identification of the band immunoprecipitated by the anti-MBP monoclonal antibody 105-120. 1: band 75 kD immunoprecipitated by the anti-MBP monoclonal antibody 105-120 from biotinylated human monocytes; 2: band 75 kD recognised by a human anti-CD64 monoclonal antibody; 3: no band detected by a control IgG1 isotype.

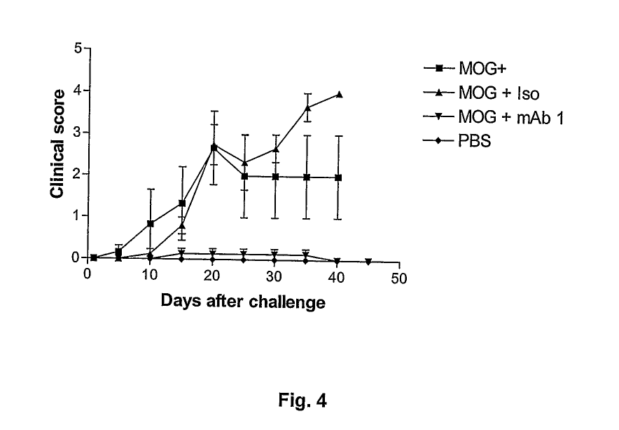

FIG. 4: Mean daily clinical score of Chronic Experimental Autoimmune Encephalitis (CEAE) induced in C57BL/6 mice with MOG 35-55. The values are expressed as mean±standard error (ES) of three independent experiments in which three mice were used for each group (n=12). Deceased mice were not included in the calculation of the score. MOG+=mice with MOG 35-55-induced CEAE; MOG+iso=mice with MOG 35-55-induced CEAE treated with human myeloma IgM (iso, 500 μg/mouse); MOG+Mab 1=mice with MOG 35-55-induced CEAE treated with the anti-MBP monoclonal antibody 105-120 (500 μg/mouse); PBS=mice treated with PBS, without CEAE. The treatment with myeloma IgM and with the anti-MBP monoclonal antibody 105-120 was carried out on day 0, 7 and 10 after the induction of CEAE.

### MATERIALS AND METHODS

### 1—Patients

After obtaining informed consent, samples of venous blood were collected from 60 patients with defined MS (7), 22 patients with other inflammatory and non inflammatory neurological diseases (2 with Guillain-Barré, 1 with myelitis, 4 with neurolupus, 7 with amyotrophic lateral sclerosis, 1 with second motor neuron syndrome, 3 with chronic polyneuropathy, 3 with cerebrovascular diseases, 1 with olivopontocerebellar) and 20 normal healthy individuals, homogeneous by gender and age.

The patients were defined as affected by stable MS when they had not had clinically documented relapses according to Poser (7) for at least one year, and with the absence of gadolinium positive MRI lesions of the encephalon. No MS patient, at the time of the blood drawing, had received corticosteroids or immunosuppressants for at least one year.

### 2—Isolation of the B Cell Clones and of Anti-Myelinic Protein Monoclonal Antibodies

The mononucleated cells of the peripheral blood were isolated by centrifuging on density gradient on Ficoll-Hypaque in accordance with standard procedures (8). Said cells were transformed with the supernatant of the marmoset cell line B95.8 infected with Epstein-Barr virus (EBV) and placed in culture in a medium containing RPMI 1640, 10% bovine fetal serum, cyclosporin A (5 μg/ml) and human interleukin-6 (5 ng/ml). After two weeks in the culture medium, the lines were prevalently characterised by B cells (CD20⁺) and after two more weeks they were able to secrete IgM. The total IgM and IgG were measured in the supernatant of the EBV⁺ lines by a previously described ELISA (5). The IgG were found to be absent. To ascertain the monoclonality of the IgM, the kappa and lambda light chains were measured by ELISA using anti-kappa and lambda light chain polyclonal antibodies of human immunoglobulins conjugated with peroxidase (Dako, Copenhagen, Denmark). In the supernatants of the lines producing monoclonal IgM, were sought monoclonal IgM against the following myelinic protein: myelin basic protein (MBP), proteolipid protein (PLP) having the following sequence:

(Seq ID No 3)
MGLLECCARC LVGAPFASLV ATGLCFFGVA LFCGCGHEAL
TGTEKLIETY FSKNYQDYEY LINVIHAFQY VIYGTASFFF
LYGALLLAEG FYTTGAVRQI FGDYKTTICG KGLSATVTGG
QKGRGSRGQH QAHSLERVCH CLGKWLGHPD KFVGITYALT
VVWLLVFACS AVPVYIYFNT WTTCQSIAFP SKTSASIGSL
CADARMYGVL PWNAFPGKVC GSNLLSICKT AEFQMTFHLF
IAAFVGAAAT LVSLLTFMIA ATYNFAVLKL MGRGTKF

and myelin oligodendrocyte glycoprotein (MOG) having the following sequence:

(Seq ID No 4)
MASLSRPSLP SCLCSFLLLL LLQVSSSYAG QFRVIGPRHP
IRALVGDEVE LPCRISPGKN ATGMEVGWYR PPFSRVVHLY
RNGKDQDGDQ APEYRGRTEL LKDAIGEGKV TLRIRNVRFS
DEGGFTCFFR DHSYQEEAAM ELKVEDPFYW VSPGVLVLLA
VLPVLLLQIT VGLVFLCLQY RLRGKLRAEI ENLHRTFDPH
FLRVPCWKIT LFVIVPVLGP LVALIICYNW LHRRLAGQFL
EELRKFSSLC YKQRIKSQER ETEATRGRGG LLRDHIPRGK
EELESLGGGK TPPGR

An ELISA was used. Briefly, in the micro-plate wells peptides of the MBP, PLP and MOG (the sequences are shown in Table 1) at the concentration of 20 μg/ml were fixed in the buffer NaCl 0.15 M-Na₂ HPO₄ pH 7.3 overnight at 4° C. and after various rinses with PBS-Tween 0.05%, the plates were blocked with PBS-bovine serum albumin (BSA) at 3% for 1 h at 37° C.

TABLE 1
Panel of the synthetic MBP, MOG and PLP peptides used

| | |
|---|---|
| Antibody | Sequence |

PBM

| | | |
|---|---|---|
| Peptide 1 (aa 38-52 of Seq ID No 1) | Mab 2 | ILDSIGRFFGGDRGA |
| Peptide 2 (aa 53-67 of Seq ID No 1) | Mab 3 | PKRGSGKDSHHPART |
| Peptide 3 (aa 68-82 of Seq ID No 1) | | AHYGSLPQKSHGRTQ |
| Peptide 4 (aa 83-99 of Seq ID No 1)* | | DPVVHFFKNIVTPRT |
| Peptide 5 (aa 105-120 of Seq ID No 1)** | Mab 1 | GKGRGLSLSRFEWGAE |
| Peptide 6 (aa 121-135 of Seq ID No 1) | | GQRPGFGYGGRASDY |
| Peptide 7 (aa 130-144 of Seq ID No 1) | | GRASDYKSAHKGFKG |

MOG

| | | |
|---|---|---|
| Peptide 1 (aa 30-49 of Seq ID No 4) | | GQFRVIGPRHPIRALVGDEV |
| Peptide 2 (aa 64-84 of Seq ID No 4 ) | Mab 4 | MEVGWYRPPFSRVVHLYRNGK |
| Peptide 3 (aa 110-129 of Seq ID No 4) | | VTLRIRNVRFSDEGGFTCFF |
| Peptide 4 (aa 120-139 of Seq ID No 4) | | SDEGGFTCFFRDHSYQEEAA |

PLP

| | | |
|---|---|---|
| Peptide 1 (aa 140-152 of Seq ID No 3) | Mab 5 | HCLGKWLGHPDKF |
| Peptide 2 (aa 185-200 of Seq ID No 3) | | QSIAFPSKTSASIGSL |
| Peptide 3 (aa 191-210 of Seq ID No 3) | | SKTSASIGSLCADARMYGVL |

PBM = Myelin Basic Protein,
MOG = Myelin Oligodendrocyte Glycoprotein,
PLP =Proteolipid Protein
*aa 84-85 were deleted,
**aa 116 (S) was substituted by E.

All peptides were synthesised by PRIMM, Milan, Italy, and purity was checked with high pressure liquid chromatography and with mass spectrometry. Subsequently, to the micro-plates were added the supernatants of the lymphoblastoid lines diluted 1:5 in PBS-ABS 0.5%, and after various washes were added rabbit IgG antibodies anti-human kappa or lambda light chains (1:10000) (Dako) and subsequently a goat IgG antibody anti-rabbit IgG conjugated with peroxidase. The colorimetric reaction was developed with o-phenyldiamine 0.1% in citrated buffer pH 4.5 and the related absorbance was read at the ELISA photometer (BIO-RAD Model 550) at 492 nm. The lines with optical density >3.0 were considered positive and cloned. Cloning was carried out by limit dilution at low cellular density (0.3 cells/well) in 60-well Terasaki micro-plates (Falcon, Bedford, Mass.) in accordance with a previously described method (9). The clones with a kappa/lambda ratio >7 are considered positive. The anti-MBP, PLP and MOG peptide monoclonal IgM were isolated and purified from the supernatants by affinity chromatography by means of Hi Trap IgM purification columns (Amersham Pharmacia, Uppsala, Sweden) according to the manufacturer's instructions and after sterile filtration they were used in the cell proliferation tests or in the in vivo experiments with animals. The mean level of concentration of the IgM is in the 100-130 μg range and all monoclonal antibodies are of the IgMκ type. The positive clones were kept in culture for many months and weekly assayed for the production of the specific monoclonal antibodies.

### 3—T Cell Lines

T cell lines sensitised to MBP, PLP and MOG were isolated from the peripheral blood of MS patients according to a method described previously (10). Before being used in the proliferation tests, the lines were subjected to at least two stimulation “pulses” with the specific peptide and with irradiated autologous monocytes and expanded with recombinant human IL-2 (10 U/ml).

### 4—Proliferation Tests

The proliferation tests were performed by incubation of 1×10⁴ MBP⁺-T cells with 10⁵ mononucleated cells as cells exhibiting the antigen previously irradiated at 3000 rad and with the monoclonal antibody IgM anti-MBP 105-120 peptide (25 μg/ml) for 72 h at 37° C. Human myeloma IgM (Calbiochem, San Diego, Calif.) was used as control monoclonal antibody. In some experiments, the anti-MBP 105-120 peptide antibody was replaced by an anti-MOG 64-84 (which correspond to the mouse anti-MOG 35-55) or anti-PLP 140-152 or anti-MBP 38-52 or anti-MBP 53-67 IgM monoclonal antibody at the same concentration. After incubation, the cells were trypsinised with trypsin 0.05%-EDTA 0.02% at 37° C. and then the proliferation tested with a method with MTT described previously (11). In some experiments, a murine anti-CD64 monoclonal antibody (Ancell, Bayport, Minn., USA) at the concentration of 50 μg/ml and a neutralising monoclonal antibody anti-human IL-10 (5 μg/ml) (R&D Systems, Minneapolis, Minn., USA). In one experiment, the conalbumin-sensitive murine T line D10.G4.1 was used at the same conditions as the human T-MBP⁺ lines was used, employing conalbumin as sensitising antigen.

The inhibition percentage was calculated according to the following formula:

$$100 - \frac{\begin{matrix}Abosorbance of stimulated and inhibited T cell line - \\ Absorbance of non stimulated T cell line\end{matrix}}{Abosrbance of stimulated and non inhibited T cell line} \times 100$$

### 5—Immunoprecipitation

1×10⁶ monocytes were marked with biotin-7-NHS (0.2 mg/ml) (Roche Diagnostics) in 50 mM borate buffer pH 8.0 for 30 min at ambient temperature (t.a.) with slow agitation. After washings with Tris buffer pH 8.3, the cells were incubated in PBS-EDTA 0.2% pH 7.4 for 45 min at t.a., washed and then lysated with the extraction buffer (Sodium borate 50 mM-NaCl 150 mM-NP40 2%-PMSF 2 mM-aprotinin 1% pH 8.0) in ice for 30 min. After centrifuging, the proteins of the pellet were measured with Lowry's method and immunoprecipitated with a complex formed by A-Sepharose Protein (Amersham-Pharmacia) previously conjugated with a rabbit polyclonal antibody anti-human IgM or anti-mouse IgG (Dako) or with the anti-MBP peptide monoclonal antibody or human myeloma IgM; in some experiments, a mouse monoclonal antibody anti-human CD64 and a mouse monoclonal antibody anti-human MHC beta chain Class II were used (Ancell, Bayport, Minn., USA). After incubation in a rotator overnight at 4° C., the immunocomplex formed was boiled at 100° C. for 5 min, then made to run in a 10%-SDS-Tricin polyacrylamide gel and transferred by electrical field onto a nitrocellulose membrane. After blocking with PBS-ABS 3%, the membrane was exposed to streptavidin conjugated with peroxidase (1:5000), and the protein bands revealed with con 4-chloro naphthol 0.075% in Tris-HCl 0.05 M pH 6.8 and 0.008% H₂O₂. In some experiments, after western blotting, the membrane was incubated with an anti-human CD64 monoclonal antibody or a control mouse IgG1 isotype (Ancell) and the bands revealed as described previously.

### 6—Interleukin-10 Measurement

1×10⁵ monocytes, non stimulated or stimulated with lipopolysaccharide (LPS) (5 μg/ml), are exposed for 24 h at 37° C. to the human anti-MBP monoclonal antibody (106-120) (25 μg/ml) or to a murine monoclonal antibody anti-human CD64 (50 μg/ml) or a control murine IgG1 isotype (25 μg/ml). The levels of interleukin-10 (IL-10) were measured in the supernatants of the cultures with a commercial ELISA sandwich using a pair of antibodies constituted by a capture anti-human IL-10 monoclonal antibody and an anti-human IL-10 monoclonal antibody conjugated with biotin. Human recombinant IL-10 was used as standard (all antibodies and the recombinant IL-10 were supplied by R&D Systems). The ELISA sensitivity limit was 7.8 pg/ml. The test was carried out according to the manufacturer's instructions.

### 7—Model of Chronic Experimental Autoimmune Encephalitis

A chronic model of experimental autoimmune encephalitis (EAE) was induced in 2-month old female mice of the C57BL/6 strain (Charles River Laboratories Italia, Calco, Italy) with the peptide of murine MOG 35-55 in accordance with a previously described method (12). The experiments were conducted with four groups of animals: The first one was injected subcutaneously (s.c.) with the MOG peptide in complete Freund's adjuvant (CFA); the second one with the MOG peptide in CFA and treated intraperitoneally (i.p.) with the human monoclonal antibody DM1A2C1 (500 μg IgM/mouse), the third one with the MOG peptide and treated with human myeloma IgM (500 μg IgM/mouse, i.p.); the fourth one with CFA and treated with PBS. The treatment with the human monoclonal antibody DM1A2C1 (Mab1) or human myeloma IgM was carried out on day 0 and repeated on days 7 and 10 after MOG injection. The mice were weighted and blind monitored daily according to the following clinical scale: 0: no clinical sign; 1: loss of tail muscle tone; 2: flaccid tail; 3: paralysis of rear limbs; 4: paralysis of the rear limbs and of the rear part of the torso; 5; paralysis of front and rear limbs; 6: death.

Histological analysis was performed on the brains and spine marrow removed from mice anesthetised with chloralium hydrate (0.4 mg/g of body weight) and perfused with paraformaldehyde at 4% in PBS in a mean period of 38 days from the induction of encephalitis. The nerve tissue was fixed in formalin 10% and parafinated; 6 nm sections were coloured with the standard hematoxylin-eosin method to evaluate inflammatory infiltrates and 9 nm sections were coloured with the fast blue Luxol method to analyse myelin. The perivascular inflammatory infiltrates (at least 5 cells/vessel) were blind counted with a morphometric grid and expressed as number/mm². The demyelination areas were measured with a morphometric grid and expressed in mm².

### 8—Statistical Analysis

For all statistical analyses, the comparison between the groups was carried out with the Mann-Whitney non parametric test. P<0.05 was considered statistically significant.

### Results

### a) Isolation of Clones Producing Anti-PBM, MOG and PLP Monoclonal IgM.

Lymphoblastoid lines were cloned from 60 patients with Multiple Sclerosis (MS), 22 patients with other inflammatory and non inflammatory neurological diseases (OND) and 20 healthy individuals (NHS). In these clones, the production of monoclonal IgM directed against different epitopes of the MBP, comprising the sequence between aa 38 to aa 144 of Seq ID No 1 of the isoform 18.5 kD of the protein, of the myelin oligodendrocyte glycoprotein (MOG) comprising the sequences between aa 30 to aa 49, aa 64 to aa 84, aa 110 to aa 129 and aa 120 to aa 139 of seq ID No 4 (which contain the immunodominant T epitopes), and of the proteolipid protein (PLP) comprising the sequences between aa 140 to aa 152 and aa 185 to aa 219 (containing the immunodominant T epitopes) was verified. The clones producing IgM with a kappa/lambda ratio >7 were considered positive. The dominant epitope for the MBP was found to correspond to the sequence between aa 105 to aa 120 of Seq ID No 1 (19/60 MS patients vs. 3/22 OND and 1/20 NHS; P=0.02)). The dominant epitope for the MOG corresponded to the sequence between aa 64 to aa 84 of Seq ID No 4 (12/60 MS vs. 5/22 OND and 1/20 NHS; n.s.). The dominant epitope for the PLP was found to correspond to the sequence between aa 140 to aa 152 of Seq ID No 3 (11/60 MS vs. 1/22 OND and 1/20 NHS; n.s.).

### b) Purification and Characterisation of the Immunomodulating Properties of the Monoclonal IgM.

The monoclonal IgM were purified by affinity chromatography from the supernatants of clones B isolated from 5 MS patients, 3 of whom were in a stable phase of the disease and 2 in progressive phase (Table 2).

TABLE 2
B cell clones producing anti-myelin protein
monoclonal IgM from patients with MS

| | | Antibody | |
|---|---|---|---|
| Clone | epitope B | name | MS clinical course |
| DM1A2C1 | PBM 105-120 | Mab 1 | Stable for 10 years |
| | | | (from the last relapse) |
| MR10C4A10A6 | PBM 38-52 | Mab 2 | Stable for 7 months |
| | | | (from the last relapse) |
| SM9F5E8D2 | PBM 53-67 | Mab 3 | Secondary-progressive |
| | | | form |
| BLU5F2E6E9 | MOG 64-84 | Mab 4 | Secondary-progressive |
| | | | form |
| GG5A3A3C6 | PLP 140-152 | Mab 5 | Stable for 6 months |
| | | | (from the last relapse) |

The mean concentration of IgM obtained oscillated between 100 and 130 μg/ml.

To assay immunomodulating capabilities, the effect of these antibodies on the inhibition of the proliferation of human T lymphocyte cell lines sensitised to MBP and to ovalbumin (OVA) as control antigen (Tables 3, 4), MOG and PLP (Table 5) was analysed. The anti-MBP (105-120) IgM, Mab1, produced by a clone (DM1A2C1) of an MS patient in long stable phase for 10 years almost entirely inhibit the proliferation of T lines sensitised to whole MBP, whereas a human myeloma IgM (Iso) used as control antibody was found to be completely ineffective. Moreover, Mab1 totally inhibits the proliferation of lines sensitised to ovalbumin (Table 3).

TABLE 3
Inhibition of the proliferation of human
MBP⁺-T lines by the monoclonal anti-MBP IgM, Mab 1.

| | | |
|---|---|---|
| T | 0.100 ± 0.002 | — |
| T + PBM (25 μg/ml) | 1.161 ± 0.05 | — |
| T + PBM + Mab 1 | 0.107 ± 0.008 | 99.4 |
| T + PBM + Iso | 1.143 ± 0.05 | 11 |
| T + OVA (25 μg/ml) | 0.659 ± 0.03 | — |
| T + OVA + Mab 1 | 0.101 ± 0.01 | 100 |

T = non stimulated MBP⁺-T line; PBM = Myelin Basic Protein; OVA = ovalbumin, Mab 1 = anti-MBP (105-120) monoclonal IgM Iso = human myeloma IgM.
The results are expressed in optical density (O.D.) as mean ± standard error of at least 3 independent experiments in each of which were used triplicate cultures (n = 9).

Other monoclonal antibodies anti-MBP 38-52 (Mab2) and 53-67 (Mab3) inhibit only partially (respectively by 76 and by 58%) the proliferation of a T cell line sensitised to the MBP (Table 4).

TABLE 4
Inhibition of the proliferation of human
MBP⁺-T lines by others anti-MBP monoclonal IgM.

| | O.D (570 nm) | % inhibition |
|---|---|---|
| T | 0.104 ± 0.03 | — |
| T + PBM (25 μg/ml) | 1.215 ± 0.006 | — |
| T + PBM + Mab 1 | 0.119 ± 0.008 | 99 |
| T + PBM + Mab 2 | 0.393 ± 0.01 | 76 |
| T + PBM + Mab 3 | 0.613 ± 0.01 | 58 |

T = non stimulated MBP⁺-T line; PBM = Myelin Basic Protein; Mab 1 = anti-MBP (105-120) monoclonal IgM; Mab 2 = anti-MBP (38-52) monoclonal IgM; Mab 3 = anti-MBP (53-67) monoclonal IgM.
The results are expressed in optical density (O.D.) as mean ± standard error of triplicate cultures of a representative experiment (n = 3)

The IgM anti-MBP 105-120, Mab1, totally inhibit also T lines sensitised to MOG 64-84 and to PLP 140-152 (Table 5).

TABLE 5
Inhibition of the proliferation of human MOG⁺-T and
PLP⁺ lines by the anti-MBP monoclonal IgM

| | O.D (570 nm) | % inhibition |
|---|---|---|
| T | 0.110 ± 0.01 | — |
| T + MOG (25 μg/ml) | 0.688 ± 0.015 | — |
| T + MOG (25 μg/ml) + Mab 1 | 0.112 ± 0.012 | 99.7 |
| T + PLP (25 μg/ml) | 0.543 ± 0.03 | — |
| T + PLP (25 μg/ml) + Mab 1 | 0.102 ± 0.012 | 100 |

T = non stimulated T line; Mab 1 = anti-MBP (105-120, 25 μg/ml).
The results are expressed in optical density (O.D.) as mean ± standard error of triplicate cultures of a representative experiment (n = 3)

The data show that the Mab1 antibody acts on antigen-dependent proliferation. Anti-MOG 64-84 (Mab4) and anti-PLP 140-152 (Mab5) monoclonal IgM, do not inhibit the MBP⁺-T lines (Table 6).

TABLE 6
Inhibition of the proliferation of human MBP⁺-T
lines by anti-MOG and -PLP monoclonal antibodies

| | O.D (570 nm) | % inhibition |
|---|---|---|
| T | 0.110 ± 0.01 | — |
| T + PBM (25 μg/ml) | 1.188 ± 0.04 | — |
| T + PBM + Mab 4 | 1.156 ± 0.05 | 3 |
| T + PBM + Mab 5 | 0.930 ± 0.02 | 22 |

T = non stimulated MBP⁺-T line; Mab 4 = anti-MOG monoclonal IgM (25 μg/ml); Mab 5 = anti-PLP monoclonal IgM (25 μg/ml).
The results are expressed in optical density (O.D.) as mean ± standard error of at least 2 independent experiments in each of which were used triplicate cultures (n = 6).

The inhibitory effect of the anti-MBP IgM is concentration- and time-dependent (FIG. 1A, 1B). Moreover, the mAb DM1A2C1, Mab1, is able to completely inhibit also the proliferation of the murine T line D10.G4.1, sensitised to conalbumin suggesting a possible in vivo activity of the human antibody in mice (Table 7).

TABLE 7
Inhibition of the proliferation of the murine D10.G4.1 T
cell line by the anti-MBP (105-120) monoclonal antibody

| | O.D (570 nm) | % inhibition |
|---|---|---|
| T | 0.086 ± 0.01 | — |
| T + CONA (25 μg/ml) | 1.284 ± 0.07 | — |
| T + CONA + Mab 1 | 0.126 ± 0.01 | 100 |
| T + CONA + Iso | 1.266 ± 0.04 | 1.5 |

T = non stimulated T line; CONA = Conalbumin; Mab 1 = anti-MBP (105-120) monoclonal IgM (25 μg/ml); Iso = human myeloma IgM.
The results are expressed in optical density (O.D.) as mean ± standard error of triplicate cultures of a representative experiment (n = 3).

### c) Identification of the Epitope Recognised by the Monoclonal Antibody

To clarify whether the immunomodulating effect could depend on a direct action on the T lines or on the cells presenting the antigen (CPA), inhibition experiments on T lines were repeated after putting the T lines or the CPA in contact with the anti-MBP IgM. It was demonstrated that the inhibitory effect of the monoclonal IgM is observed only after putting the IgM in contact with the CPA but not with the human T cells (Table 8).

TABLE 8
Identification of the target cell recognised
by the anti-MBP (105-120) monoclonal antibody

| | O.D (570 nm) | % inhibition |
|---|---|---|
| T | 0.137 ± 0.008 | — |
| T + PBM (25 μg/ml) | 1.257 ± 0.04 | — |
| Mab 1 + MNC | 0.147 ± 0.008 | 99.2 |
| Mab 1 + T + PBM | 1.233 ± 0.023 | 2 |
| T + PBM + Iso | 1.243 ± 0.015 | 1 |

T = non stimulated human T⁺ line; PBM = Myelin Basic Protein; MNC = Monocytes; Mab 1 = anti-MBP (105-120) Mab; Iso = human myeloma IgM; Mab 1 + MNC = monocytes preincubated with the Mab 1 and then used in the T lines proliferation test; PBM + T + Mab 1 = human T line preincubated with the Mab 1 and then stimulated with the MBP and used in the proliferation tests.
The results are expressed in optical density (O.D.) as mean ± standard error of triplicate cultures of a representative experiment (n = 3).

The data demonstrate that the monoclonal antibody acts directly on the CPA, intervening in the antigen presentation process.

To exclude that the monoclonal antibody could bind to the class II MHC and to determine whether it recognised any protein on the monocytes other than the class II MHC, T lymphocytes and monocytes, previously biotinylated with the anti-MBP monoclonal antibody were immunoprecipitated with a monoclonal antibody directed against the chain of the human antigens HLA DR, DQ and DP and with human myeloma IgM as control and the immunoprecipitated proteins were separated by electrophoresis on polyacrylamide gel in SDS and Western blotting. The monoclonal antibody immunoprecipitated from the non activated monocytes, but not from the T lymphocytes, a protein of about 74-75 kD, sharply distinct from the immunoprecipitated proteins of the myeloma IgM and from the class II anti-MHC antibody. An identical pattern was obtained after immunoprecipitation of the proteins of murine splenocyte membrane (FIG. 2). These results suggest that the immunomodulating action of said antibody does not interfere on the antigen recognition process by the class II antigen, but takes place through an action on the monocytes.

After consulting the Swiss Prot protein database, two monocyte surface proteins of about 75 kD were identified: the TNFr (CD120b) and “the high affinity immunoglobulin gamma Fc receptor I precursor” (Fc-gamma RI, CD64). The FASTA program was then used to highlight any sequence homology with the epitope comprised in the sequence between aa 105 and aa 120 of the MBP recognised by our monoclonal IgM. While no significant homologies emerged between the TNFr and the MBP (105-120), a homology did emerge with the Fc-gamma RI (CD64) regarding 4 consecutive amino acids and two others, contiguous and corresponding to the sequence 222-228 of the extracellular domain. The homologous sequence is the following:

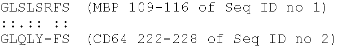

To demonstrate whether said homology could justify the cross-reactivity of the Mab1 antibody with the CD64, the protein 75 kD of the biotinylated monocytes was immunoprecipitated with the monoclonal antibody Mab1 and the proteins separated by electrophoresis on polyacrylamide gel and subsequent immunoblotting with an anti-human CD64 IgG1 monoclonal antibody (Ancell, USA) and an IgG1 control isotype. The protein 75 kD is revealed by the anti-CD64 but not by the IgG1 isotype (FIG. 3), demonstrating that the band 75 kD, immunoprecipitated by the mAb DM1A2C1 (Mab1) is recognised by the anti-CD64 mAb. Moreover, growing concentrations of the peptide 222-228 CD64 (GLQLYFS) but not of a “scrambled” peptide (QFLGSYL Seq ID no 5) inhibit the binding of mAb DM1A2C1 (Mab1) to the MBP (105-120), indicating a full homology between the two epitopes.

Moreover, the inhibitory effect of the mAb DM1A2C1 and of the anti-CD64 mAb is completely abolished in the presence of the peptide 222-228 CD64 but not of the “scrambled” peptide (Table 9).

TABLE 9
Identification of the proliferation of human
MBP⁺-T cells by the anti-MBP monoclonal IgM
in the presence of the peptide CD64 (222-228).

| | O.D (570 nm) | % inhibition |
|---|---|---|
| T | 0.109 ± 0.009 | — |
| T + PBM (25 μg/ml) | 1.209 ± 0.04 | — |
| T + PBM + Mab 1 | 0.116 ± 0.004 | 99.4 |
| T + PBM + Mab 1 + pep 1 | 1.237 ± 0.015 | 0 |
| T + PBM + Mab 1 + pep 2 | 0.133 ± 0.003 | 98 |
| T + PBM + Mab CD64 | 0.101 ± 0.006 | 100 |
| T + PBM + Mab CD64 + pep 1 | 1.205 ± 0.01 | 0.4 |
| T + PBM + Mab CD64 + pep 2 | 0.129 ± 0.009 | 98.4 |

T = non stimulated T line; Mab 1 = anti-MBP (105-120) Mab; CD64 = anti-CD64 monoclonal antibody; pep 1 = peptide CD64 (222-228); pep 2 = “scrambled” peptide.
The results are expressed in optical density (O.D.) as mean ± standard error of triplicate cultures of a representative experiment (n = 3).

The data demonstrates that the epitope between aa 222 and aa 228 of CD64 is the specific epitope involved in the in vitro inhibitory activity of mAb 1.

### d) Action Mechanism

To identify the possible action mechanism of the in vitro immunosuppressive activity of the mAb DM1A2C1, we investigated whether said activity is due to the stimulation of the production of Interleukin-10 (IL-10) by the monocytes. The inhibitor effects of the mAb DM1A2C1 and of the anti-CD64 mAb on a MBP-sensitive T line isolated from an MS patient are completely abolished in the presence of a neutralising anti-IL-10 mAb (Table 10).

TABLE 10
Inhibition of the proliferation of human MBP⁺-T
line by an by the anti-IL-10 monoclonal IgM antibody.

| | O.D (570 nm) | % inhibition |
|---|---|---|
| T | 0.118 ± 0.015 | — |
| T + PBM (25 μg/ml) | 1.189 ± 0.035 | — |
| T + PBM + Mab 1 | 0.142 ± 0.022 | 98 |
| T + PBM + Mab 1 + Mab IL-10 | 1.253 ± 0.009 | 0 |
| T + PBM + Mab 1 + Iso IgG1 | 0.131 ± 0.016 | 1 |
| T + PBM + Mab CD64 | 0.141 ± 0.028 | 98 |
| T + PBM + Mab CD64 + Mab IL-10 | 1.226 ± 0.017 | 0 |
| T + PBM + Mab CD64 + Iso IgG1 | 0.129 ± 0.022 | 99 |

T = non stimulated T line; Mab 1 = anti-MBP (105-120) Mab; Mab CD64 = anti-CD64 monoclonal antibody; Mab IL-10 = anti-IL-10 monoclonal antibody; iso IgG1 = control IgG1 isotype.
The results are expressed in optical density (O.D.) as mean ± standard error of triplicate cultures of a representative experiment (n = 3).

The author also demonstrated high levels of IL-10 in the supernatants of monocytes stimulated with the mAb DM1A2C1 (Mab1) or with the anti-CD64 mAb but not with an IgG1 isotype. These levels were further increased when the monocytes were activated with lipopolysaccharide (LPS) (Table 11).

TABLE 11
Levels of IL-10 in the supernatants of human monocytes
incubated with anti-MBP (105-120) antibody

| | (pg/ml) |
|---|---|
| MNC | 49 ± 2.8 |
| MNC + Mab 1 | 4062 ± 68* |
| MNC + Mab CD64 | 3953 ± 48 |
| MNC + iso IgG1 | 57 ± 5 |
| MNC + LPS | 1059 ± 20 |
| MNC + LPS + Mab 1 | 6538 ± 52^{§} |
| MNC + LPS + Mab CD64 | 6362 ± 39 |
| MNC + LPS + iso IgG1 | 1002 ± 27 |

MNC = non stimulated monocytes; Mab 1 = anti-MBP (105-120) Mab; Mab CD64 = anti-CD64 monoclonal antibody; iso IgG1 = control IgG1 isotype; LPS = lipopolysaccharide.
The results are expressed in optical density (O.D.) as mean ± standard error of triplicate cultures of a representative experiment in which each point was analysed in duplicate (n = 6).
*P = 0.002 vs. MNC or vs. MNC + Iso IgG1,
^{§}P = 0.002 vs. MNC + LPS or vs. MNC + LPS + Iso IgG1.

These data demonstrate that the in vitro immunosuppressive activity of the mAb DM1A2C1, Mab1, is exercised by the simulation of the production and release by the monocytes of high quantities of IL-10, a cytokine with known anti-inflammatory activity.

### e) In Vivo Immunosuppressive Activity

The homology of the human peptide 222-228 CD64 with the residues 231-237 of the murine Fcgamma RI receptor and the inhibitory activity of the mAb1 on the proliferation of the murine T line D10.G4.1 sensitised to conalbumin suggested the possibility of using the human mAb DM1A2C1 in a model of chronic EAE induced with the MOG protein in C57BL/6 mice. None of the mice treated preventively with the mAb 1 developed the disease for a long observation period (about 40 days) unlike the mice pre-treated with human myeloma IgM used as control antibody, which developed a severe disease with some dead animals (FIG. 4).

The histological examination of the brains and of the spine marrow did not identify inflammatory or demyelination foci in the chronic EAE mice treated with the mAb 1. In contrast, inflammatory foci were identified in the brains and spine marrow of the chronic EAE mice treated with the human myeloma IgM which are significantly more numerous with respect to untreated chronic EAE mice (Table 12).

TABLE 12
Perivascular lymphocyte infiltrates in the brains and spine
marrow of chronic experimental autoimmune encephalitis

| | Brains | Spine marrow |
|---|---|---|
| | (number/mm²) | (number/mm²) |
| MOG⁺ (n = 15) | 2 ± 0.28 | 1.67 ± 0.25 |
| MOG + Iso (n = 15) | 3.3 ± 0.27*^{§} | 3.7 ± 0.30*^{§§} |
| MOG + Mab 1 (n = 15) | 0.07 ± 0.06 | 0 ± 0 |
| PBS (n = 15) | 0.06 ± 0.06 | 0.06 ± 0.06 |

MOG⁺ = untreated mice with chronic EAE; MOG + iso = mice with chronic EAE (CEAE) treated with human myeloma IgM (iso) (500 μg/mouse); MOG + Mab 1 = mice with chronic EAE treated with the anti-MBP (105-120) monoclonal antibody (500 μg/mouse); PBS = mice without chronic EAE treated with PBS.
The results are expressed as number of infiltrates/mm². The number in parentheses indicates the total number of sections analysed (at least five sections per brain or marrow).
*P < 0.0001 vs. PBS and MOG + Mab 1,
^{§}P = 0.0049 vs. MOG⁺,
^{§§}P = 0.0002 vs. CEAE.

Moreover, in the chronic EAE mice treated with the mAb 1, DM1A2C1, no demyelination areas were identified in the brain and spine marrow. On the other hand, the demyelination areas in the brains of EAE mice treated with myeloma IgM were significantly higher than in the untreated EAE mice (Table 13).

TABLE 13
Demyelination areas in the brains and spine marrow
of chronic experimental autoimmune encephalitis

| | Brains | Spine marrow |
|---|---|---|
| | (number/mm²) | (number/mm²) |
| MOG⁺ | 0.064 ± 0.005 | 0.077 ± 0.005 |
| | (n = 30) | (n = 20) |
| MOG + Iso | 0.09 ± 0.005* | 0.096 ± 0.005 |
| | (n = 40) | (n = 35) |
| MOG + Mab 1 (n = 0) | 0 ± 0 | 0 ± 0 |
| PBS (n = 0) | 0 ± 0 | 0 ± 0 |

MOG⁺ = untreated mice with chronic EAE; MOG + iso = mice with chronic EAE treated with human myeloma IgM (iso) (500 μg/mouse); MOG + Mab 1 = mice with chronic EAE treated with the anti-MBP (105-120) monoclonal antibody (500 μg/mouse); PBS = mice without chronic EAE treated with PBS.
The results are expressed as mm². N = number of analysed areas,
*P = 0.0028 vs CEAE.

### CONCLUSIONS

The present results demonstrate the existence of an IgM anti-PBM (105-120) monoclonal antibody isolated from MS patients in a long stable phase of the disease, with powerful in vitro immunosuppressive properties directed against clones sensitised to myelinic proteins and in vivo in a model of chronic EAE induced by the MOG protein which is considered one of the animal experimental models that can be best superposed to human MS. The epitope recognised by the antibody is the residue 222-228 of the extracellular domain of the CD64 on the monocytes. The immunosuppressive activity takes place through a binding with said molecule on the surface of the monocytes, which are the most efficient cells exhibiting the antigen in the peripheral blood, inducing the production and release of considerable quantities of IL-10, a cytokine with a well known anti-inflammatory activity. The natural antibody has immunosuppressive properties mediated through an absolutely new mechanism, different from the other immunosuppressants (azathioprine and cyclophosphamide) used in the therapy of MS which act on the cycle of the cell DNA with potential long-term oncogenic effects or such as mitoxantrone which also has cardiotoxic effects. It therefore constitutes a new immunosuppressor drug to be used in the therapy of MS but also of other immunomediated and inflammatory diseases of the nervous system, and in general in organ-specific autoimmune diseases (Lupus, rheumatoid arthritis, thyroiditis, etc.).

Other immunomediated pathologies of the nervous system could be susceptible of a potential treatment with the anti-MBP monoclonal antibody; for example, but without limitation, post-infective and post-vaccine autoimmune encephalomyelitis, including acute disseminated encephalomyelitis (ADEM). Furthermore, the immunosuppressor antibody can be used in all those neurological diseases in which classic immunosuppressants (azathioprine and cyclophosphamide) such as, by way of non limiting examples: chronic inflammatory polyneuropathies intractable to treatment with corticosteroids and/or immunoglobulins e.v., multifocal motor neuropathy; myasthenia that do not respond to treatment with corticosteroids, anti-cholinesterase drugs and immunosuppressants. Lastly, this new immunosuppressor can be used in transplant rejection pathologies, in other organ-specific autoimmune diseases (autoimmune thyroiditis, Lupus, rheumatoid arthritis). In many of these pathologies, immunosuppressants are used that are often used in MS as well.

### BIBLIOGRAPHY

1—Compston A., Coles A. Lancet 359, 1221-1231 (2002).
2—Poser S., Wikstrom J., Bauer H. J. J. Neurol. Sci. 40, 159-168 (1979).
3—Thompson A. J., Hutchinson M., Brazil J., Feighery C., Martin E. A. Q. J. M. 225, 69-80 (1986).
4—Hawkins S. A., McDonnell G. V. J. Neurol. Neurosurg. Psychiatry 67, 148-152 (1999).
5—Annunziata P., Pluchino S., Martino T., Guazzi G. J. Neuroimmunol. 77, 128-133 (1997).
6—Gonsette R. E. Mult. Scler. 1, 306-312 (1996).
7—Poser C. M., Paty D. W., Scheinberg L., McDonald W. I., Davis F. A., Ebers G. C., Johnson K. P:, Sibley W. A., Silberg D. H., Tourtellotte W. W. Ann. Neurol. 13. 227-231 (1983).
8—Boyum A. Scand. J. Clin. Lab. Invest. Suppl., 97, 77-89, 1968
9—Jingwu Z., Henderikx P., Ying C., Medaer R., Raus J. C. M. J. Immunol. Methods 123, 153-154 (1989).
10—Burns J., Rosenzweig A., Zweiman B., Lisak R. P. Cell. Immunol. 77, 363-371 (1983). 98-
11—Hussain R. F., Nouri A. M., Oliver R. T. S. Immunol. Methods 160, 89-96 (1993).
12—Mendel I., Kerlero de Rosbo N., Ben-Nun A. Eur. J. Immunol. 25, 1951-1959 (1995).

1. An antibody, recombinant or synthetic fragments thereof able to recognise and bind at least one epitope of the myelin basic protein.

2. The antibody, recombinant or synthetic fragments thereof as claimed in claim 1, able to recognize also an epitope of the protein of the class of Fc receptors (FcR).

3. The antibody, recombinant or synthetic fragments thereof as claimed in claim 2, wherein the protein of the class of Fc receptors(FcR) is the protein FcRI (CD64).

4. The antibody, recombinant or synthetic fragments thereof as claimed in claim 1 wherein the epitope of the myelin basic protein is comprised in the amino acid region from aa 105 to aa 120 of SEQ ID No 1.

5. The antibody, recombinant or synthetic fragments thereof as claimed in claim 4 wherein the epitope of the myelin basic protein essentially consists of the amino acid region from aa 109 to aa 116 of SEQ ID No 1.

6. The antibody, recombinant or synthetic fragments thereof as claimed in claim 3 wherein the epitope of the protein CD64 essentially consists of the amino acid region from aa 222 to aa 228 of SEQ ID No 2.

7. The antibody, recombinant or synthetic fragments thereof as claimed in claim 1 being of human origin or humanized.

8. The antibody, recombinant or synthetic fragments thereof as claimed in claim 7 for medical use.

9. A pharmaceutical composition for the prevention and/or treatment of autoimmune diseases comprising the antibody, recombinant or synthetic fragments thereof as claimed in claim 7 in therapeutically effective amounts and appropriate dilutants, and/or excipients and/or vehicles.

10. The pharmaceutical composition as claimed in claim 9 for the prevention and treatment of multiple sclerosis.

11. A nucleic acid encoding the antibody, recombinant or synthetic fragments thereof as claimed in claim 1.

12. A recombinant expression vector comprising the nucleic acid as claimed in claim 11 able to express the antibody, recombinant or synthetic fragments thereof able to recognise and bind at least one epitope of the myelin basic protein.

13. The antibody, recombinant or synthetic fragments thereof as claimed in claim 2 being of human origin or humanized.

14. The antibody, recombinant or synthetic fragments thereof as claimed in claim 3 being of human origin or humanized.

15. The antibody, recombinant or synthetic fragments thereof as claimed in claim 4 being of human origin or humanized.

16. The antibody, recombinant or synthetic fragments thereof as claimed in claim 5 being of human origin or humanized.

17. The antibody, recombinant or synthetic fragments thereof as claimed in claim 6 being of human origin or humanized.

18. A nucleic acid encoding the antibody, recombinant or synthetic fragments thereof as claimed in claim 2.

19. A nucleic acid encoding the antibody, recombinant or synthetic fragments thereof as claimed in claim 3.

20. A nucleic acid encoding the antibody, recombinant or synthetic fragments thereof as claimed in claim 4.
